(19) United States
(12) Patent Application Publication
(10) Pub. No.: US 2025/0269175 A1
(43) Pub. Date: Aug. 28, 2025

(54) DELIVERY AND RETRIEVAL SYSTEM FOR A MEDICAL DEVICE

(71) Applicant: Medtronic, Inc., Minneapolis, MN (US)

(72) Inventors: Zhongping C. Yang, Woodbury, MN (US); Thomas A. Anderson, New Hope, MN (US); Ronald A. Drake, St. Louis Park, MN (US); Brian P. Colin, Anoka, MN (US); Lester O. Stener, Hudson, WI (US)

(21) Appl. No.: 18/858,167

(22) PCT Filed: Apr. 25, 2023
(86) PCT No.: PCT/IB2023/054228

§ 371 Date: Oct. 18, 2024

Related U.S. Application Data
(60) Provisional application No. 63/364,015, filed on May 2, 2022.

(51) Int. Cl. A61N 1/05; A61N 1/375

(52) U.S. Cl. CPC A61N 1/057; A61N 1/3756; A61N 2001/0578

(57) ABSTRACT

A medical system configured to impart a torque to a medical device within a patient. The medical system includes a driver including a driver body supporting a driver cup. An intermediate member is configured to position within a cup recess of the driver cup and a device recess of the medical device when the medical device resides within a cup volume of the driver cup. The intermediate member is configured to transfer a torque from the driver cup to the medical device to cause a rotation of the medical device. In examples, the medical device includes an attachment member configured to engage or disengage tissue when the medical device rotates. The medical system may include a delivery catheter configured to deliver and/or retrieve the driver cup, intermediate member, and medical device.

### TECHNICAL FIELD

This disclosure is related to system for delivery and/or retrieval of implantable medical devices.

### BACKGROUND

Various types of implantable medical devices have been implanted for treating or monitoring one or more conditions of a patient. Such implantable medical devices may be adapted to allow medical devices to monitor and/or treat conditions or functions relating to heart, muscle, nerve, brain, stomach, endocrine organs or other organs and their related functions. The implantable medical devices may be implanted at target locations selected to detect a physiological condition of the patient and/or deliver one or more therapies. For example, implantable medical devices may be delivered to locations within an atrium or ventricle of a heart to sense intrinsic cardiac signals and deliver pacing or antitachyarrhythmia shock therapy.

Some implantable medical devices are sized to be completely implanted within one of the chambers of the heart and/or another anatomical volume of the patient to detect a physiological condition and/or deliver one or more therapies. Such implantable medical devices may utilize delivery and/or retrieval systems to allow a clinician to navigate the implantable medical device (e.g., through vasculature of the patient) to the target location, and/or to retrieve the implantable medical device from the patient. In some examples, the implantable medical device may include one or more anchoring components intended to engage tissues at the target location (e.g., for implantation) and/or disengage from tissue at the target location (e.g., for retrieval).

### SUMMARY

The disclosure describes a medical system configured to deliver, position, retrieve, and/or otherwise re-orient an implantable medical device within an anatomical volume (e.g., a chamber of a heart) within a patient. The medical system is configured to receive a torque (e.g., from a clinician) and impart the torque via a driver body to a driver cup. The driver cup defines a cup volume configured to receive at least some portion of the implantable medical device. The medical system includes an intermediate member configured to position within a cup recess of the driver cup and a device recess of the implantable medical device, to allow the driver cup to transfer a torque via the intermediate member from the driver cup to the implantable medical device. In examples, the medical system includes a delivery catheter configured to deliver and/or retrieve the driver cup, the intermediate member, and/or the implantable medical device through vasculature of the patient.

In an example, a medical system, comprises: a driver including a driver body configured to receive a torque, wherein the driver body defines a lumen and a lumen opening; a driver cup supported at a distal end of the driver body, wherein the driver body is configured to impart the torque on the driver cup, and wherein the driver cup includes a cup surface defining a cup volume configured to receive an implantable medical device, wherein the lumen opening opens into the cup volume; and an intermediate member including a member body and a transfer portion of the member body, wherein the member body is configured to slidably translate within the lumen to position the transfer portion within the cup volume, wherein the transfer portion is configured to position within a cup recess of the cup surface and a device recess of the implantable medical device when the cup volume receives the implantable medical device and the transfer portion is positioned within the cup volume, wherein the transfer portion is configured to transfer the torque from the driver cup to the implantable medical device when the transfer portion positions within the cup recess and the device recess, and wherein the driver body, the driver cup, and the intermediate member are configured to be positioned within an anatomical volume defined by a body of a patient.

In an example, a method comprises: imparting a torque, using a driver body of a driver, on a driver cup supported at a distal end of the driver body, wherein the driver body defines a lumen and a lumen opening; and transferring the torque from the driver cup defining a cup volume to an implantable medical device within the cup volume using a transfer portion of an intermediate member positioned within a cup recess and a device recess, wherein the lumen opening opens into the cup volume, wherein the cup recess is defined by the driver cup and the device recess is defined by the implantable medical device, wherein the intermediate member includes a member body including the transfer portion, wherein the member body is slidably translatable within the lumen, and wherein the driver body, the driver cup, and the intermediate member are positioned within an anatomical volume defined by a body of a patient.

In an example, a medical system comprises: a driver receptacle which is rotatable with respect to a longitudinal axis of the medical system, the driver receptacle including a receptacle surface defining a receptacle volume configured to receive at least a proximal portion of an implantable medical device; and an intermediate member including a member body and a transfer portion of the member body, wherein the member body is configured to longitudinally translate relative to the receptacle to position the transfer portion along the receptacle volume, wherein the transfer portion is configured to position within a receptacle recess of the receptacle surface when the receptacle volume receives the implantable medical device and the transfer portion is positioned within the receptacle volume, wherein the transfer portion is configured to transfer the torque from the driver receptacle to the implantable medical device when the transfer portion positions within the receptacle recess, and wherein the driver body, the driver receptacle, and the intermediate member are configured to be positioned within an anatomical volume defined by a body of a patient.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical system and delivery catheter within a heart.

FIG. 2 is a perspective diagram of an example medical system including a driver, an intermediate member, and a delivery catheter.

FIG. 3 is a cross-sectional diagram of the example medical system.

FIG. 4 is a perspective diagram of the example medical system with an implantable medical device positioned within a driver cup.

FIG. 5 is a cross-sectional diagram of the example medical system with the implantable medical device positioned within the driver cup.

FIG. 6 is a cross-sectional diagram of a transfer portion within a cup recess of a driver cup and a device recess of an implantable medical device.

FIG. 7 is a cross-sectional diagram of the transfer portion of FIG. 6 transferring a torque from the driver cup to the implantable medical device.

FIG. 8 is a perspective diagram of an example driver cup.

FIG. 9 is a cross-sectional view of an example driver cup and an intermediate member.

FIG. 10 is a schematic diagram of an intermediate member including a loop tether.

FIG. 11 is a perspective diagram of an example loop tether engaging an implantable medical device.

FIG. 12 is a cross-sectional diagram of a transfer portion defined by a loop tether within a cup recess of a driver cup and a device recess of an implantable medical device.

FIG. 13 is a cross-sectional diagram of the transfer portion defined by the loop tether of FIG. 12 transferring a torque from the driver cup to the implantable medical device.

FIG. 14 illustrates an example technique for transferring a torque to an implantable medical device.

### DETAILED DESCRIPTION

This disclosure describes a medical system configured to deliver, position, and/or retrieve an implantable medical device (“IMD”) within an anatomical volume (e.g., a chamber of a heart) within a patient. The medical system is configured to receive a torque (e.g., from a clinician) and impart the torque to IMD to implant, retrieve, re-position, and/or re-orient the IMD in the anatomical volume. In examples, the medical system is configured to enable a rotation of the IMD around a device axis when the medical system imparts the torque. The rotation of the implantable medical device may cause an attachment member of the implantable medical device to engage tissues at a target site, disengage tissues at the target site, and/or otherwise cause a re-orientation of the medical device within the patient.

The medical system includes a driver (e.g., an elongate body) configured to receive the torque (e.g., from the clinician) and impart the torque to a receptacle or cup at a distal end of the driver (“driver cup” or “driver receptacle”). The driver cup defines a cup volume configured to receive at least some portion of the implantable medical device. In examples, the driver is configured to position at least partially within vasculature of a patient when the driver cup is positioned within the anatomical volume. In some examples, the driver includes a proximal portion configured to be extracorporeal to a patient and a distal portion configured to be intracorporeal to the patient. The distal portion may support the driver cup. The medical system may be configured such that a torque imparted to the proximal portion (e.g., by a clinician) causes the distal portion to impart the torque to the driver cup. The medical system is configured to transfer the torque from the driver cup to the IMD to implant, retrieve, re-position, and/or re-orient the IMD. In examples, the IMD includes an attachment member (e.g., a helix) configured to engage tissue or disengage from tissue based on a rotation of the IMD. The medical system may transfer the torque from the driver cup to the IMD to cause the IMD rotation, such that the attachment member engages with or disengages from the tissue.

The medical system includes an intermediate member configured to transfer the torque from the driver cup to the IMD when the cup volume receives the IMD. The intermediate member is configured to position substantially between the driver cup and the IMD when the cup volume receives the IMD, such that when the driver cup imparts a torque on the intermediate member, the intermediate member imparts the torque to the IMD. In examples, the intermediate member is configured to position within a cup recess defined within the cup volume and a device recess defined by the IMD when the intermediate member positions between the driver cup and the IMD. The cup recess may be configured such that a portion of the intermediate member protrudes from the cup recess when the intermediate member positions in the cup recess. The protruding portion may reside within the device recess of the IMD, such that the intermediate portion receives the torque from the cup recess and transfers the torque to the device recess, thereby transferring the torque to the IMD. The device recess may be formed by, for example, a proximal portion of the IMD, a retrieval structure of the IMD, or some other portion of the IMD.

The driver (e.g., a driver body) defines a lumen (“driver lumen”) and a driver lumen opening which opens into the cup volume defined by the driver cup. The intermediate member is configured to slidably translate within the driver lumen, such that a clinician may cause the intermediate member to translate proximally and/or distally within the driver lumen, and/or cause the driver and the driver cup to translate relative to the intermediate member. The relative translation may assist in positioning the intermediate member within the cup recess and the device recess, such that the intermediate member may transfer the torque from the driver cup to the IMD. The intermediate member may include a proximal portion configured to be extracorporeal to a patient and a distal portion configured to be intracorporeal to the patient (e.g., within the driver lumen).

For example, the intermediate member may be configured to extend through the cup volume and distally beyond the driver cup, such that the intermediate member may be positioned within a device recess of an IMD (e.g., an implanted IMD) distal to the driver cup. The driver and driver cup may be distally advanced over the intermediate member to cause the cup volume to receive the IMD and the positioned intermediate member. The driver cup may be configured to cause the intermediate member to position within the cup recess when the cup volume receives the IMD, such that the intermediate member is positioned in both the device recess and the cup recess. In some examples, the intermediate member may be extended into the cup volume, and the driver cup may be configured to receive the IMD with the intermediate member extended within the cup volume. The driver cup may be configured to rotate relative to the IMD, such that the rotation causes the intermediate member to position (e.g., slot into) the cup recess and/or the device recess, such that the intermediate member is positioned in both the device recess and the cup recess. With the intermediate member positioned in the cup recess and the device recess, the intermediate member may transfer a torque imparted to the driver cup (e.g., by the driver) to the IMD.

The use of the intermediate member may ease any mechanical coupling requirements that might exist between the driver cup and the IMD in order to torque the IMD using a cup. For example, in some systems a cup may be configured as a socket intended to mechanically mate with a proximal end of an IMD to transfer a torque. The cup may define recesses configured to receive protrusions defined by the IMD, and/or define protrusions configured to insert into recesses defined by the IMD. Transfer of a torque from the cup to the IMD may thus require relatively precise alignment between the cup and the IMD to achieve the necessary mechanical mating. Achieving this relatively precise alignment may present difficulty when the cup and the device are positioned within a constrained anatomical space of a patient (e.g., a chamber of a heart).

In contrast, use of the intermediate member to transfer torque received from a cup recess to a device recess may alleviate this alignment burden. For example, the cup recess and/or the device recess may be relatively larger than the intermediate member, easing the difficulty of positioning the intermediate member in the cup recess and/or the device recess. The larger cup recess may be configured such that a cup recess bearing surface substantially drives the intermediate member into a device recess bearing surface when the driver cup rotates relative to the IMD, such that the intermediate member transfers the torque from the driver cup to the IMD despite the larger size of the cup recess and/or the device recess relative to the intermediate member. The relatively large sizes of the cup recess and/or the device recess relative to the intermediate member may ease any alignment requirements that might exist between the driver cup and the IMD for the transfer of a torque.

The medical system may be configured such that the intermediate member substantially causes an alignment between the cup recess and the device recess. In examples, a cup surface defining the cup volume may be configured such that a rotation of the cup surface (e.g., by rotating the driver cup) around a portion of the IMD (e.g., a proximal portion) causes the intermediate member to slip between the cup surface and the IMD until the intermediate member inserts (e.g., slots) into the cup recess and the device recess. For example, rotation of the driver cup relative to the IMD may cause the intermediate member to slip between the cup surface and the IMD until the intermediate member slots into the cup recess, with subsequent rotation of the driver cup and the slotted intermediate member causing a protruding portion of the intermediate member to slot into the device recess, such that the intermediate member may transfer torque from the driver cup to the IMD. Alternately, rotation of the driver cup relative to the IMD may cause the intermediate member to slip between the cup surface and the IMD until the intermediate member slots into the device recess, with subsequent rotation of the driver cup and the device-slotted intermediate member causing a portion of the intermediate member to slot into the cup recess, such that the intermediate member may transfer torque from the driver cup to the IMD. Thus, the use of the intermediate member to transfer torque from the driver cup to the IMD may ease alignment burdens on a clinician desiring to transfer torque from the driver cup to the IMD.

The medical system may include a delivery catheter including a delivery cup or receptacle. The delivery cup may define a delivery cup volume configured to receive the driver cup, the intermediate member, and at least a portion of the IMD. The delivery catheter may define a delivery catheter lumen and a delivery lumen opening which opens to the delivery cup volume. The driver may be configured to slidably translate within the delivery catheter lumen and through the delivery lumen opening, such that relative movement between the driver and the delivery catheter may cause the driver cup, the intermediate member, and/or at least the portion of the IMD to position within the delivery cup volume and/or exit the delivery cup volume (via the delivery lumen opening). The delivery catheter may be configured to transition through the vasculature of a patient, such that the driver cup, the intermediate member, and/or the IMD may be retrieved from and/or delivered to an anatomical volume of the patient (e.g., a heart chamber).

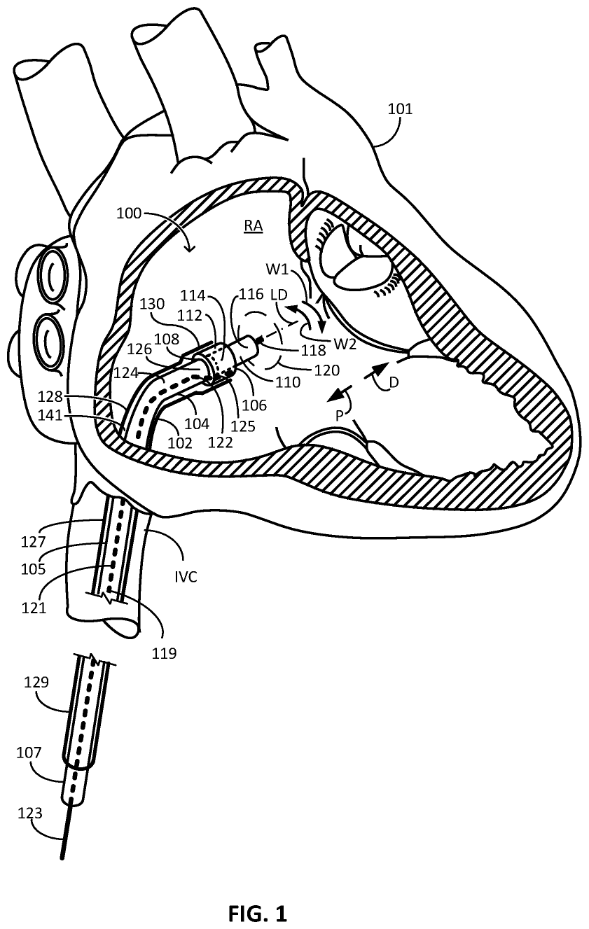

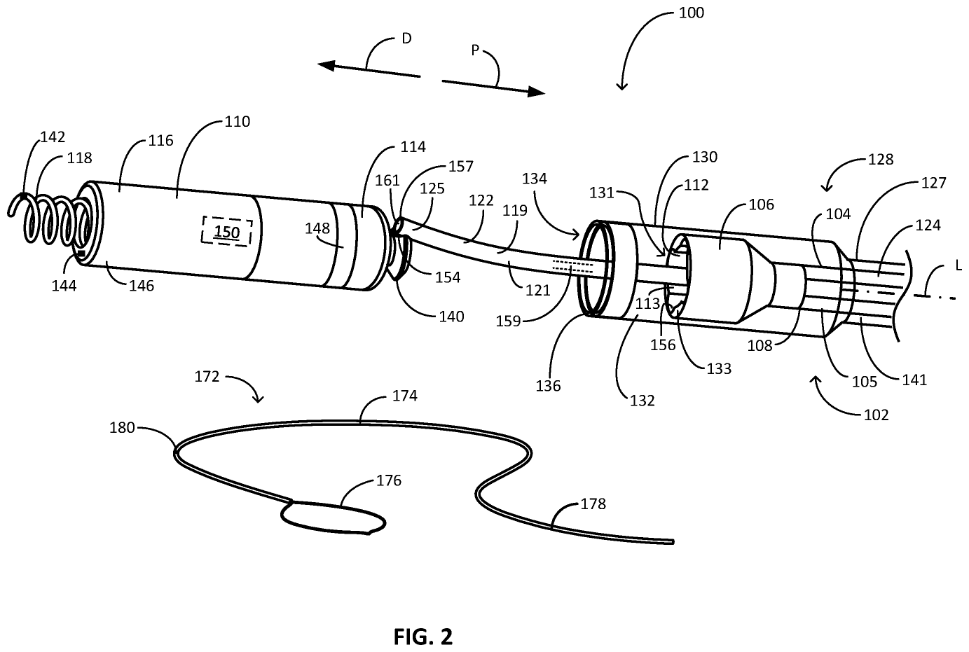

FIG. 1 is a conceptual diagram illustrating an example medical system 100 within a right atrium (“RA”) of a heart 101. Medical system 100 includes driver 102 including driver body 104. Driver body 104 may include a distal portion 105 (“driver body distal portion 105”) configured to be intracorporeal to the patient and a proximal portion 107 (“driver body proximal portion 107”) which may be extracorporeal to the patient when driver body distal portion 105 is intracorporeal. A driver cup 106 is supported at a distal end 108 of driver body 104 (“driver body distal end 108”). In some examples, driver cup 106 and driver body 104 may be substantially separate components. In some examples, driver cup 106 may be substantially contiguous with driver body 104, such that driver cup 106 and driver body 104 define a unified component. An IMD 110 is positioned within (e.g., inserted into) a cup volume 112 defined by driver cup 106. In the example of FIG. 1, a proximal portion 114 of IMD 110 (“IMD proximal portion 114”) is positioned within cup volume 112. IMD 110 may include a distal portion 116 (“IMD distal portion 116”) opposite IMD proximal portion 114. In examples, driver cup 106 includes a cup surface (e.g., cup surface 113 (FIGS. 2, 3, 6-9)) defining cup volume 112.

In examples, IMD 110 (e.g., IMD distal portion 116) supports an attachment member 118 configured to engage tissue within a target site 120 of an anatomical volume. Attachment member 118 may be supported in IMD distal portion 116. In some examples, attachment member 118 is configured (e.g., as a helix) such that rotation of IMD 110 about a device axis LD defined by IMD 110 causes attachment member to engage and/or disengage tissues with target site 120. For example, attachment member 118 may be configured such that rotation of IMD 110 in a first rotational direction W1 about device axis LD causes attachment member 118 to engage (or alternately, disengage from) tissues within target site 120. Attachment member 118 may be configured such that rotation of IMD 110 about device axis LD in a second rotational direction W2 substantially opposite first rotational direction W1 causes attachment member 118 to disengage from (or alternately, engage) tissues within target site 120. In examples, IMD 110 includes one or more components (e.g., a communication antenna, a sensor, or another component) configured to rotate around and or revolve about device axis LD when IMD 110 rotates about device axis LD. Medical system 100 may cause IMD 110 to rotate about device axis LD to cause one or more of the components to substantially establish a specific orientation with respect to the anatomy of the patient, a another device implanted within or worn by the patient, another device external to the patient, and/or other devices.

Driver body 104 is configured to receive a torque (e.g., from a clinician) and transfer the torque to driver cup 106. Medical system 100 includes an intermediate member 122 configured to position within cup volume 112 between driver cup 106 and IMD proximal portion 114 to transfer the torque from driver cup 106 to IMD proximal portion 114. Intermediate member 122 includes a body 119 (“member body 119’). Member body 119 may be a substantially elongate body. In examples, intermediate member 122 (e.g., member body 119) includes a transfer portion 125 (e.g., a distal end portion of member body 119) configured to position within cup volume 112 between driver cup 106 and IMD proximal portion 114 to transfer the torque. Intermediate member 122 may position within a cup recess (e.g., cup recess 156 (FIGS. 2-9, 12, 13)) defined by driver cup 106 within cup volume 112 and a device recess (e.g., device recess 154 (FIGS. 2-7, 12, 13)) defined by IMD 110 (e.g., IMD proximal portion 114). Intermediate member 122 is configured such that, when intermediate member 122 receives a torque from driver cup 106 (e.g., from a surface defining the cup recess), and intermediate member 122 is positioned with the device recess, intermediate member 22 imparts the torque to the IMD (e.g., to a surface defining the device recess). Hence, medical system 100 is configured to mechanically couple driver cup 102 and IMD 110 using at least intermediate member 22, such that a torque imparted to driver body 104 may cause rotation of IMD 110 in first rotational direction W1 and/or second rotation direction W2.

Driver 102 (e.g., driver body 104) defines a lumen 124 (“driver lumen 124”) and a lumen opening 126 (“driver lumen opening 126”) at driver body distal end 108. Driver lumen opening 126 opens into cup volume 112. Intermediate member 122 (e.g., member body 119) is configured to slidably transfer within driver lumen 124 such that intermediate member 122 may translate relative to driver body 104 and driver cup 106 (and vice-versa) in a distal direction D and/or a proximal direction P. The relative translation between intermediate member 122 and driver body 104 and/or driver cup 106 may assist in positioning intermediate member 122 within the cup recess and/or the device recess. In examples, medical system 100 is configured such that, when intermediate member 122 is between driver cup 106 and IMD 110 (e.g., IMD proximal portion 114), rotation of driver cup 106 relative to IMD 110 (e.g., by imparting a torque to driver body 104) may cause intermediate member 122 to insert into at least one of or both of the cup recess and the device recess, such that intermediate member 122 may transfer torque from driver cup 106 to IMD 110.

Intermediate member 122 may include a distal portion 121 (“intermediate member distal portion 121”) configured to be intracorporeal to the patient (e.g., within driver lumen 124) and a proximal portion 123 (“intermediate member proximal portion 123”) which may be extracorporeal to the patient when intermediate member distal portion 121 is intracorporeal. Intermediate member distal portion 121 may include transfer portion 125. In examples, member body 119 defines intermediate member distal portion 121 and intermediate member proximal portion 123

Medical system 100 may include a delivery catheter 128 configured to retrieve driver cup 106, intermediate member 122, and/or IMD 110 from an anatomical volume of the patient (e.g., the RA). In examples, delivery catheter 128 is configured to deliver driver cup 106, intermediate member 122, and/or IMD 110 to an anatomical volume of the patient. Delivery catheter 128 is illustrated as transparent in FIG. 1 for clarity. Delivery catheter 128 may include a distal portion 127 (“delivery catheter distal portion 127”) configured to be intracorporeal to the patient and a proximal portion 129 (“delivery catheter proximal portion 129”) which may be extracorporeal to the patient when delivery catheter distal portion 127 is intracorporeal. In examples, delivery catheter 128 is configured to deliver and/or retrieve driver cup 106, intermediate member 122, and/or IMD 110 using vasculature of a patient, such as an IVC or other vasculature leading to the anatomical volume.

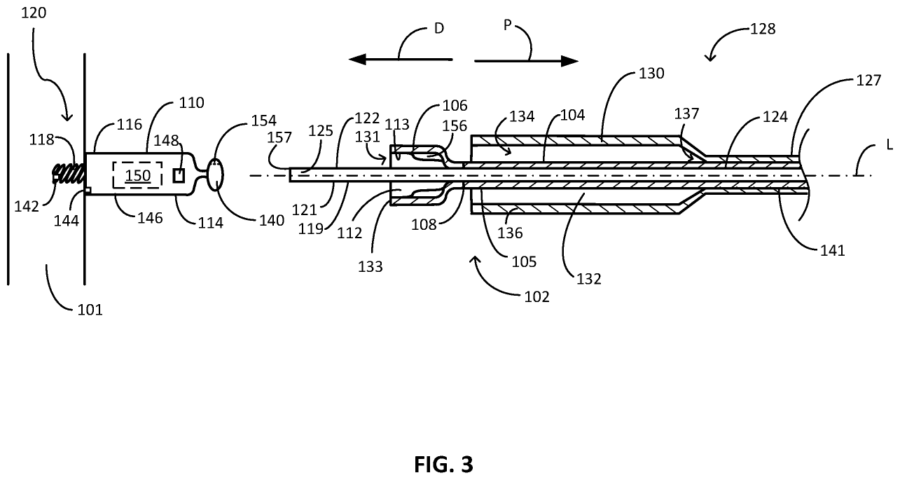

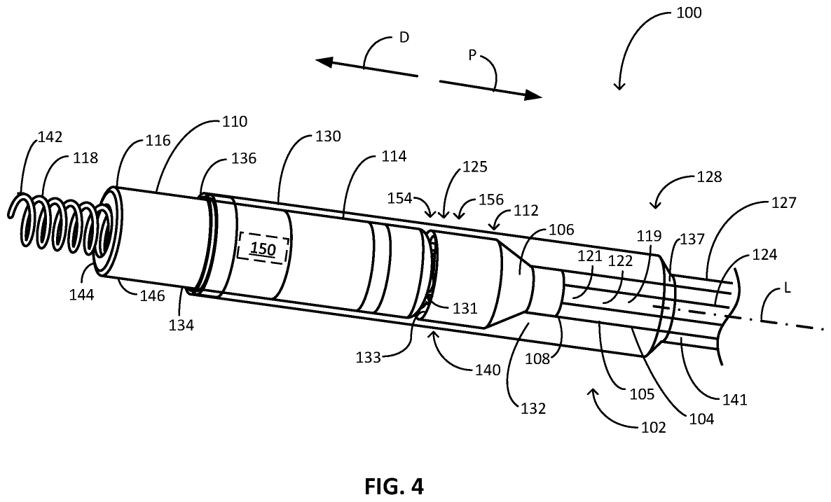

In examples, delivery catheter 128 includes a delivery cup 130 or receptacle defining a delivery cup volume (e.g., delivery cup volume 132 (FIGS. 2-5, 11)) configured to receive driver cup 106, intermediate member 122, and at least a portion of IMD 110. Delivery catheter 128 may define a lumen 141 (“delivery catheter lumen 141”) and a delivery lumen opening (e.g., delivery lumen opening 137 (FIGS. 3-4)) which opens to delivery cup volume 132. At least driver body 104 may be configured to slidably translate within delivery catheter lumen 141 and through the delivery lumen opening such that relative movement between driver body 104 and delivery catheter 128 may cause relative movement between driver cup 106, intermediate member 122, and/or IMD 110 and delivery catheter 128. Delivery cup 130 may define an opening (e.g., delivery cup opening 134 (FIGS. 3, 4)) at a distal end of delivery cup 130 (e.g., delivery cup distal end 136, FIGS. 2-5). The delivery cup opening may be configured such that driver cup 106, intermediate member 122, and at least a portion of IMD 110 may pass therethrough.

Delivery catheter 128 may be configured to retrieve IMD 110 when IMD 110 is anchored to tissues within target site 120 (e.g., anchored by attachment member 118). For example, delivery catheter 128 may be configured to transition through vasculature of a patient to position driver cup 106 and intermediate member 122 in the proximity of IMD 110 when IMD 110 is anchored to tissues within target site 120. Delivery catheter 128 may be configured such that a force in the distal direction D on intermediate member 122 (e.g., by a clinician) causes intermediate member 122 to extend distal to the delivery cup opening of delivery cup 130 (e.g., toward IMD 110). Delivery catheter 128 may be configured such that a force in the distal direction D on driver body 104 (e.g., exerted by a clinician) causes driver cup 106 to extend distal to the delivery cup opening of delivery cup 130 (e.g., toward IMD 110). Delivery catheter 128 (e.g., delivery cup 130) may be configured to receive driver cup 106, intermediate member 122, and a portion of IMD 110 when intermediate member 122 is positioned between driver cup 106 and IMD 110. For example, delivery catheter 128 may move in the distal direction D relative to driver cup 106, intermediate member 122, and IMD 110 to receive driver cup 106, intermediate member 122, and at least the portion of IMD 110. Delivery catheter 128 may be configured to remove driver cup 106, intermediate member 122, and IMD 110 from an anatomical volume of the patient (e.g., the RA) once, for example, driver cup 106 and intermediate member 122 have imparted a torque to IMD 110 causing attachment member 118 to disengage from tissues within target site 120. Delivery catheter 128, driver cup 106, intermediate member 122, and IMD 110 may subsequently be withdrawn from the patient (e.g., via vasculature of the patient).

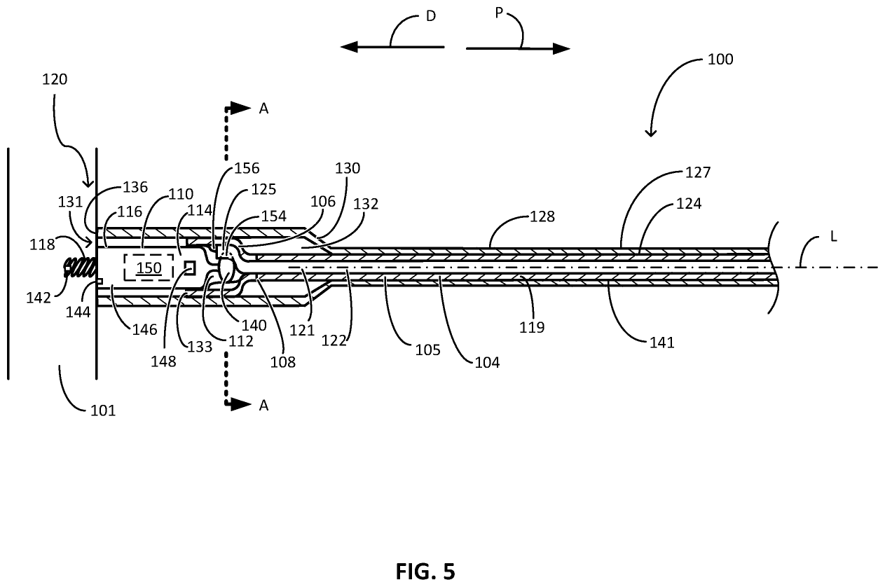

In examples, delivery catheter 128 is configured to position IMD 110 in proximity to target site 120 such that IMD 110 may be anchored to tissues within target site 120 (e.g., anchored by attachment member 118). For example, delivery catheter 128 may be configured to position driver cup 106 and intermediate member 122 within the delivery cup volume when IMD 110 is positioned within cup volume 112 and intermediate member 122 is positioned between driver cup 106 and IMD 110 (e.g., when intermediate member 122 is positioned within cup recess 156 and device recess 154 (FIGS. 5-7, 12, 13)). Delivery catheter 128 may be configured to traverse vasculature of the patient to position IMD 110 (e.g., attachment member 118) within or in proximity to target site 120. Medical system 100 (e.g., driver body 104, driver cup 106, and intermediate member 122) may impart a torque to IMD 110 to cause attachment member 118 to engage tissues (e.g., tissue within target site 120) when attachment member 118 is within or in proximity to target site 120. Medical system 100 may be configured such that a force in the proximal direction P on driver body 104 causes driver cup 106 and/or intermediate member 122 to disengage from IMD 110 as IMD 110 remains anchored to tissues within or in proximity to target site 120. Delivery catheter 128, driver cup 106, and intermediate member 122 may subsequently be withdrawn from the patient (e.g., via vasculature of the patient).

Although the examples herein discuss delivery, retrieval, and/or positioning of IMD 110 within the RA of heart 101, medical system 100 may be configured to position IMD 110 in any of the other chambers of heart 101 and/or in other anatomical volumes of a patient in a like manner as that described for the RA of heart 101. Further, although the examples herein discuss attachment member 118 defining a helix, attachment member 118 may defines other structures, such as one or more elongated tines extending from, for example, IMD distal portion 116. Target site 120 may include an appendage of the RA, or the triangle of Koch region of the RA, or some other portion of heart 101, or some other location within a body of a patient.

FIG. 2 illustrates a perspective view of medical system 100 with intermediate member 122 extended distal to driver cup 106 and delivery cup 130 of delivery catheter 128. Driver cup 106 defines an opening 131 (“driver cup opening 131”) opening into cup volume 112. In examples, a distal end 133 of driver cup 106 (“driver cup distal end 133”) defines driver cup opening 131. In FIG. 2, driver cup 106 is positioned within a cup volume 132 defined by delivery cup 130 (“delivery cup volume 132”). Delivery catheter 128 and driver body 104 are presented as transparent in FIG. 2 for clarity. FIG. 3 illustrates a cross-sectional view of medical system 100 taken along a cutting plane parallel to a longitudinal axis L defined by driver body 104, with intermediate member 122 extended distal to driver cup 106 and driver cup 106 extended distal (via driver body 104) distal to delivery cup 130.

Driver cup opening 131 is configured (e.g., sized) such that cup volume 112 may receive IMD 110 via driver cup opening 131. For example, driver cup opening 131 may be sized such that at least a portion of IMD 110 (e.g., IMD proximal portion 114) may pass through driver cup opening 131 in the proximal direction P when cup volume 112 receives IMD 110 (e.g., IMD proximal portion 114). In examples, driver cup opening 131 is configured (e.g., sized) such that IMD 110 may exit cup volume 112 via driver cup opening 131. For example, driver cup opening 131 may be sized such that IMD 110 (e.g., IMD proximal portion 114) may pass through driver cup opening 131 in the distal direction D when IMD 110 exits cup volume 112 (e.g., when driver cup 106 is proximally withdrawn from IMD 110 by driver body 104).

Delivery cup 130 defines an opening 134 (“delivery cup opening 134”) at a distal end 136 of delivery cup 130 (“delivery cup distal end 136”) which opens into delivery cup volume 132. Delivery cup opening 134 is sized such that intermediate member 122, driver cup 106, at least some portion of driver body distal portion 105, and/or at least IMD proximal portion 114 may pass in the proximal direction P and/or in the distal direction D through delivery cup opening 134. In examples, delivery catheter 128 defines a lumen 141 (“delivery catheter lumen 141”) opening into delivery cup volume 132. Delivery catheter lumen 141 is configured such that at least driver body 104 may translate within delivery catheter lumen 141 in the distal direction D and/or the proximal direction P.

Driver body 104 may translate (e.g., within delivery catheter lumen 141) in the distal direction D and/or the proximal direction P relative to intermediate member 122, delivery catheter 128, and/or IMD 110. Driver body 104 may be configured such that a force exerted on driver body 104 (e.g., by a clinician) causes a translation of driver body 104 within delivery catheter lumen 141, and the translation of driver body 104 causes a translation of driver cup 106. Driver body 104 may be configured to alter a position of driver cup 106 relative to intermediate member 122, delivery catheter 128, and/or IMD 110. For example, driver cup 106 may be moved (e.g., by a clinician exerting a force on driver body 104) in the distal direction D substantially toward IMD 110 to cause cup volume 112 to receive IMD proximal portion 114 (and/or intermediate member 122). Driver cup 106 may be moved (e.g., by a clinician exerting a force on driver body 104) in the proximal direction P substantially away from IMD 110 to cause IMD 110 (and/or intermediate member 122) to exit cup volume 112. Driver body 104 may be configured to translate within delivery catheter lumen 141 such that driver cup 106 and/or other portions of driver body 104 positions distal to and/or proximal to either or both of delivery cup opening 134 and/or a distal end 157 of intermediate member 122 (“intermediate member distal end 157”). For example, driver cup 106 may position distal to delivery cup opening 134 (as depicted in FIG. 3) and/or may position proximal to delivery cup opening 134 (as depicted in FIGS. 2, 4, 5).

IMD 110, which in some examples can comprise a pacemaker such as a leadless and/or wholly intracardiac pacemaker, may include one or more electrodes such as electrode 142 supported by attachment member 118, electrode 144 supported by a housing 146 of IMD 110 (“IMD housing 146”), and/or electrode 148 (e.g., a return electrode) supported by IMD housing 146. One or more of electrodes 142, 144, 148 may be electrically connected to operating circuitry 150. Operating circuitry 150 may be configured to deliver therapy to a patient and/or sense physiological signals of the patient using electrodes 142, 144, 148. In examples, at least a portion of operating circuitry 150 is supported by IMD housing 146. In some examples, at least a portion of operating circuitry 150 is supported by another device displaced from IMD 110, such as another device within the patient and/or another device extracorporeal to the patient. In examples, IMD 110 (e.g., IMD proximal portion 114) includes a retrieval structure 140 configured to engage with medical system 100 and/or another medical device to, for example, implant IMD 110 within an anatomical volume, retrieve IMD 110 from an anatomical volume, and/or otherwise re-orient IMD 110 when IMD 110 is positioned within an anatomical volume.

As discussed, intermediate member 122 is configured to translate (e.g., within driver lumen 124) in the distal direction D and/or the proximal direction P relative to driver body 104, driver cup 106, delivery catheter 128, and/or IMD 110. The relative translation of intermediate member 122 allows a position of transfer portion 125 to be altered relative to driver cup 106 and/or IMD 110, such that transfer portion 125 may be positioned within a device recess 154 defined by IMD 110 (e.g., IMD proximal portion 114 and/or retrieval structure 140) and/or a cup recess 156 defined by driver cup 106 (e.g., defined by cup surface 113). For example, Intermediate member 122 may be configured such that a force exerted (e.g., by a clinician) on intermediate member proximal portion 123 (FIG. 1) causes the position of transfer portion 125 to be altered relative to driver cup 106 and/or IMD 110. Intermediate member 122 may be configured to translate within driver lumen 124 such that transfer portion 125 and/or other portions of intermediate member distal portion 121 may position distal to and/or proximal to either or both of driver cup opening 131 and/or delivery cup opening 134.

In examples, member body 119 defines a lumen 159 (“member lumen 159”). Member lumen 159 may extend at least partially through intermediate member distal portion 121 and/or intermediate member proximal portion 123. In examples, member body 119 defines an opening 161 (“member lumen opening 161”) which opens into member lumen 159 at intermediate member distal end 157. Intermediate member 122 may be configured to translate within driver lumen 124 such that member lumen opening 161 may position distal to and/or proximal to either or both of driver cup opening 131 and/or delivery cup opening 134.

FIG. 4 illustrates a perspective view of medical system 100 with intermediate member 122 and IMD 110 positioned within cup volume 112 (FIGS. 2, 3, 5), and driver cup 106, intermediate member 122, and IMD 110 positioned within delivery cup volume 132. Delivery catheter 128 and driver body 104 are presented as transparent in FIG. 4 for clarity. FIG. 5 illustrates a cross-sectional view of medical system 100 taken along the cutting plane of FIG. 3, with intermediate member 122 and IMD 110 positioned within cup volume 112, and driver cup 106, intermediate member 122, and IMD 110 positioned within delivery cup volume 132.

As depicted in FIGS. 4, 5, driver body 104 and driver cup 106 may be distally advanced (e.g., in the distal direction D) toward IMD 110 to cause cup volume 112 to receive IMD 110 (e.g., IMD proximal portion 114) and/or transfer portion 125. In examples, cup volume 112 may receive IMD 110 and transfer portion 125 when transfer portion 125 is positioned within device recess 154. Driver cup 106 may be configured to cause intermediate member 122 to position within cup recess 156 when cup volume 112 receives IMD 110, such that intermediate member 122 (e.g., transfer portion 125) is positioned in both device recess 154 and cup recess 156. For example, driver cup 106 may be configured to rotate relative to IMD 110. The rotation of driver cup 106 relative to IMD 110 (e.g., when intermediate member is positioned between driver cup 106 and IMD 110) may cause intermediate member 122 to position (e.g., slot into) cup recess 156 and/or device recess 154, such that intermediate member 122 (e.g., transfer portion 125) is positioned in both cup recess 156 and device recess 154. With intermediate member 122 positioned in cup recess 156 and device recess 154, intermediate member 122 may transfer a torque imparted to driver cup 106 (e.g., by driver body 104) to IMD 110 to, for example, cause attachment member 118 to engage with tissue, cause attachment member to disengage from tissue, and/or otherwise re-orient IMD 110.

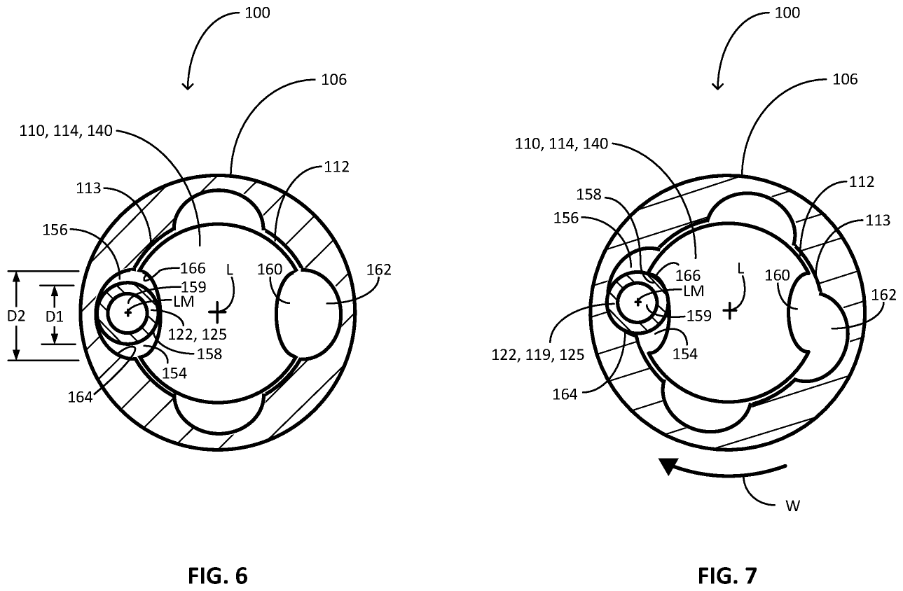

FIG. 6 illustrates a cross-sectional view of medical system 100 taken along cutting plane A of FIG. 5. Cutting plane A is substantially perpendicular to longitudinal axis L. FIG. 6 illustrates intermediate member 122 (e.g., transfer portion 125 and/or member body 119) positioned within cup recess 156 of driver cup 106 and positioned within device recess 154 of IMD 110. In examples, device recess 154 is defined by IMD proximal portion 114 and/or retrieval structure 140. IMD 110 (e.g., IMD proximal portion 114 and/or retrieval structure 140) is positioned within cup volume 112 of driver cup 106. In examples, driver cup 106 defines additional cup recesses such as cup recess 160, which may be configured similarly to cup recess 156. IMD 110 may define additional device recesses such as device recess 162, which may be configured similarly to device recess 154.

Intermediate member 122 (e.g., transfer portion 125) is positioned within cup recess 156 and device recess 154 such that when driver cup 106 imparts a torque on intermediate member 122, intermediate member 122 imparts the torque to IMD 110. In examples, driver cup 106 defines a cup recess surface 164 configured to impart a force on intermediate member 122 when driver cup 106 rotates around longitudinal axis L to impart the torque on intermediate member 122. Intermediate member 122 may be configured to exert the force on a device recess surface 166 of IMD 110 to transfer the torque from driver cup 106 to IMD 110. Cup recess surface 164 may be a portion of cup surface 113. In examples, cup recess surface 164 at least partially defines cup recess 156. Device recess surface 166 may a least partially define device recess 154. In examples, when driver cup 106 imparts a torque on intermediate member 122, transfer portion 125 imparts the torque to IMD 110.

For example, FIG. 7 illustrates driver cup 106 experiencing a torque (e.g., imparted from driver body 104 (FIG. 5) causing a rotation of driver cup 106 in a rotational direction W. The rotational direction W may be, for example, first rotational direction W1 or second rotational direction W2 (FIG. 1). The rotation of driver cup 106 causes cup recess surface 164 to contact and impart a force on intermediate member 122 (e.g., member body 119). In examples, the force imparted from cup recess surface 164 to intermediate member 122 comprises at least a force component substantially perpendicular to longitudinal axis L. In response, intermediate member 122 contacts and transmits at least some portion of the force (e.g., a torquing force) to device recess surface 166 of IMD 110. The portion of the force transmitted to device recess surface 166 causes a resultant torque on IMD 110 around longitudinal axis L. Hence, the action of intermediate member 122 (e.g., transfer portion 125) transmitting a force from cup recess surface 164 to device recess surface 166 acts to transfer a torque from driver cup 106 to IMD 110.

In examples, cup recess 156 (e.g., cup recess surface 164 defining cup recess 156) is configured to cause transfer portion 125 to revolve around (e.g., orbit) longitudinal axis L when transfer portion 125 transfers the torque from driver cup 106 to IMD 110. Transfer portion 125 may cause device recess 154 (e.g., device recess surface 166 defining device recess 154) to revolve around longitudinal axis L when transfer portion 125 revolves around longitudinal axis L. In examples, IMD 110 is configured to rotate about longitudinal axis L when transfer portion 125 and/or device recess 154 revolve around longitudinal axis L. In examples, transfer portion 125 exerts a force on device recess surface 166 comprising at least a force component substantially parallel to the force exerted by cup recess surface 164 on transfer portion 125.

Intermediate member 122, cup recess 156, and/or device recess 154 may be configured to ease any alignment between driver cup 106 and IMD 110 that might be required prior to torque transfer. In examples, intermediate member 122 is sized such that cup recess 156 and/or device recess 154 define a cross-sectional dimension larger than a cross-sectional dimension defined by intermediate member 122, such that intermediate member 122 may position concurrently within cup recess 156 and device recess 154 more easily. For example, intermediate member 122 (e.g., transfer portion 125) may define a cross-sectional dimension D1. Cup recess surface 164 may define cup recess 156 such that cup recess 156 extends over a cross-sectional dimension D2, with cross-sectional dimension D2 greater than cross-sectional dimension D1. In examples, cross-sectional dimension D2 is greater than cross-sectional dimension D1 by a factor of at least 10%, at least 20%, at least 50%, at least 100%, or at least another percentage.

Similarly, device recess surface 166 may define device recess 154 such that device recess 154 extends substantially over the cross-sectional dimension D2. In examples, cross-sectional dimension D1 is substantially parallel to cross-sectional dimension D2 when intermediate member 122 is concurrently positioned in cup recess 156 and device recess 154. In some examples, cross-sectional dimension D1 and cross-sectional dimension D2 are substantially perpendicular to a longitudinal axis LM passing through member body 119 (e.g., through member lumen 159) when intermediate member 122 is concurrently positioned in cup recess 156 and device recess 154. The relatively large sizes of cup recess 156 and/or device recess 154 relative to intermediate member 122 may ease any alignment requirements that might exist between driver cup 106 and IMD 110 in order for intermediate member 122 to transfer torque from driver cup 106 to IMD 110. In other examples, at least some portion of cup recess surface 164 and/or device recess surface 166 is configured to substantially conform to at least some portion of a surface defined by intermediate member 122 (e.g., transfer portion 145).

Member body 119 may be configured to bend and/or define curvatures (e.g., within vasculature of a patient) as well as transfer torque from driver cup 106 to IMD 110. In examples, member body 119 defines a flexible portion supporting and/or contiguous with transfer portion 125. The flexible portion may be, for example, a portion of member body 119 proximal to transfer portion 125 (e.g., one or more portions of intermediate member distal portion 121 and/or intermediate member proximal portion 123). In examples, the transfer portion defines a first stiffness and the flexible portion defines a second stiffness, wherein the first stiffness is greater than the second stiffness. The first stiffness may be indicative of an extent to which transfer portion 125 resists deformation in response to a force (e.g., a force exerted by cup recess surface 164) on transfer portion 125. The second stiffness may be indicative of an extent to which the flexible portion resists deformation in response to the force. Hence, the flexible portion (having the lower stiffness) may be configured to bend and/or define curvatures while transfer portion 125 (having the higher stiffness) transfer torque from driver cup 106 to IMD 110.

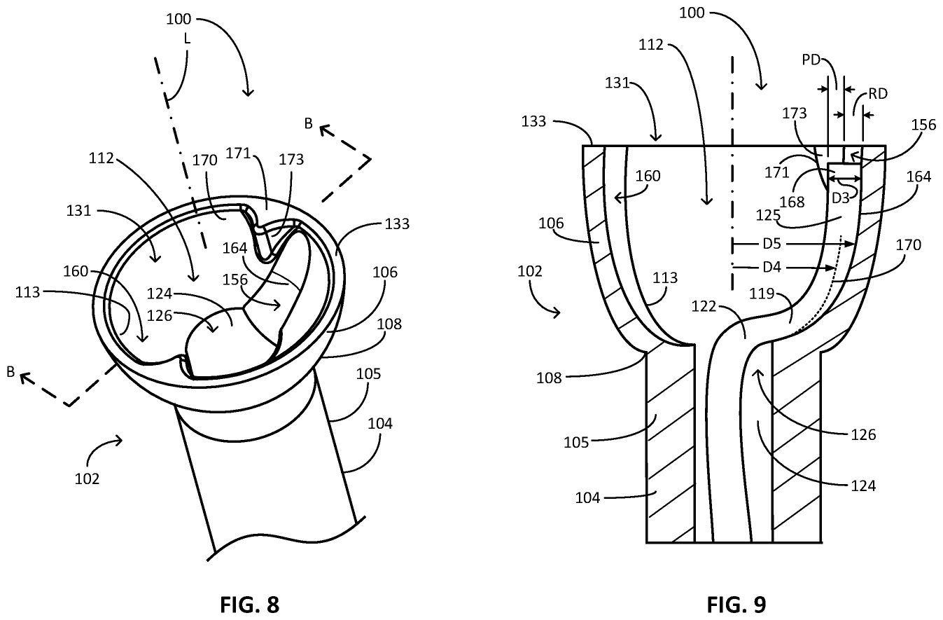

FIG. 8 illustrates a perspective view of an example driver cup 106 of medical system 100. FIG. 9 illustrates a cross-sectional view of the driver cup of FIG. 8 taken along cutting plane B of FIG. 8. FIG. 9 illustrates intermediate member 122 within driver lumen 124 and transfer portion 125 positioned within cup recess 156 and contacting cup recess surface 164.

Intermediate member 122 (e.g., transfer portion 125) and/or cup recess 156 may be configured such that a portion 168 of intermediate member 122 (“protruding portion 168”) protrudes from cup recess 156 when intermediate member 122 positions in cup recess 156 (e.g., when intermediate member 122 contacts at least some portion of cup recess surface 164). Intermediate member 122 and/or device recess 154 may be configured such that protruding portion 168 may reside within device recess 154, such that protruding portion 168 may transfer torque to device recess surface 166 when cup recess surface 164 transfers torque to intermediate member 122 (e.g., as depicted in FIG. 7).

For example, cup surface 113 (e.g., cup recess surface 164) may define cup recess 156 such that cup recess 156 extends over a recess depth RD. Recess depth RD may be substantially perpendicular to longitudinal axis L extending through cup volume 112. Intermediate member 122 may be configured to define a displacement D3 greater than the recess depth RD. Displacement D3 may be substantially perpendicular to longitudinal axis L when intermediate member 122 is in contact with cup surface 113 (e.g., cup recess surface 164). In examples, (e.g., when transfer portion 125 is substantially circular), displacement D3 may be substantially the same as the displacement D1. Intermediate member 122 may be configured such that protruding portion 158 defines a protruding displacement PD protruding from cup recess 156 when intermediate member 122 is positioned within cup recess 156 (e.g., when intermediate member 122 contacts cup surface 113).

In some examples, cup surface 113 includes a main cup surface 170 in addition to cup recess surface 164. In examples, main cup surface 170 and cup recess surface 164 form a contiguous portion of cup surface 113. For example, main cup surface 170 and cup recess surface 164 may be joined to define a cornered portion of cup surface 113, a curved portion of cup surface 113, a substantially planar portion of cup surface 113, or a portion of cup surface 113 exhibiting another surface profile. Main cup surface 170 and cup recess surface are depicted in FIGS. 8 and 9. In FIG. 9, a portion of main cup surface 170 is depicted as a hidden line (e.g., hidden by intermediate member 122 positioned within cup recess 156).

In examples, main cup surface 170 defines a fourth displacement D4 (e.g., a first radius) from longitudinal axis L and cup recess surface 164 defines a fifth displacement D5 (e.g., a second radius) from longitudinal axis L. Fourth displacement D4 may be less than fifth displacement D5. In examples, fifth displacement D5 and fourth displacement D4 (e.g., a difference between fifth displacement D5 and fourth displacement D4) define recess depth RD of cup recess 156. Fifth displacement D5 may be substantially parallel to forth displacement D4. In some examples, fifth displacement D5 and forth displacement D4 are substantially perpendicular to longitudinal axis L. In some examples, main cup surface 170 is some portion of a first surface of revolution defined about longitudinal axis L.

In some examples, driver cup 106 (e.g., main cup surface 170 and/or cup recess service 164 driver cup 106 and/or IMD 110 may be configured such that intermediate member 122 (e.g., transfer portion 125) substantially causes an alignment between cup recess 156 and device recess 154 sufficient to enable intermediate member 122 to transfer torque. For example, driver cup 106 may be configured such that a rotation of cup surface 113 (e.g., by rotating driver cup 106) around a portion of IMD 110 (e.g., IMD distal portion 116) causes intermediate member 122 to slip between cup surface 113 and IMD 110 until intermediate member 122 inserts (e.g., slots) into cup recess 156 and/or device recess 154.

For example, rotation of driver cup 106 relative to IMD 110 may cause intermediate member 122 to slip between cup surface 113 and IMD 110 until intermediate member 122 slots into cup recess 156. Subsequent rotation of driver cup 106 and the slotted intermediate member 122 may cause protruding portion 158 to slot into device recess 154, such that protruding portion 158 may transfer torque from driver cup 106 to IMD 110. Alternately, rotation of driver cup 106 relative to IMD 110 may cause intermediate member 122 to slip between cup surface 113 and IMD 110 until intermediate member 122 slots into device recess 154. Subsequent rotation of driver cup 106 and device-slotted intermediate member 122 may a portion of intermediate member 122 (e.g., a portion other than protruding portion 158) to slot into cup recess 156, such that protruding portion 158 may transfer torque from driver cup 106 to IMD 110. Hence, the use of cup surface 113, main cup surface 170, cup recess surface 164, intermediate member 122, and/or device recess 154 to transfer torque from driver cup 106 to IMD 110 may ease alignment burdens on a clinician desiring to transfer torque from driver cup 106 to IMD 110.

In examples, driver cup 106 (e.g., cup surface 113) defines a bearing structure 171 configured to guide intermediate member 122 into cup recess 156. Bearing structure 171 may include a bearing surface 173 configured to protrude from cup surface 113 (e.g., main cup surface 170) toward longitudinal axis L. In examples, bearing structure 171 is substantially adjacent cup recess 156. Bearing surface 173 may be configured to contact intermediate member 122 to cease and/or substantially prevent a relative rotation between cup surface 113 and some portion of intermediate member 122 (e.g., transfer portion 125), such that intermediate member 122 positions within cup recess 156.

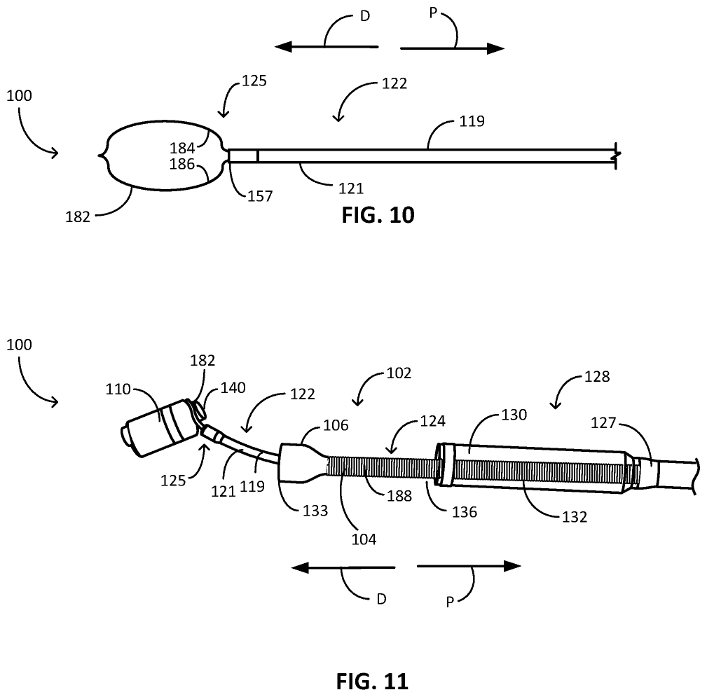

Medical system 100 may be configured to exert a proximal force on IMD 110 (e.g., a proximal force exerted by a clinician). Medical system 100 may be configured to engage IMD 110 (e.g., retrieval structure 140) to exert the proximal force on IMD 110. Medical system 100 may exert the proximal force on IMD 110 to cause cup volume 112 to receive IMD 110, and/or for other reasons. For example, medical system 100 may exert the proximal force on IMD 110 to move IMD 110 proximally relative to driver cup 106 to cause cup volume 112 to receive IMD 110. Medical system 100 may exert the proximal force on IMD 110 to assist in moving driver cup 106 distally relative to IMD 110 to cause cup volume 112 to receive IMD 110. Medical system 100 may be configured to exert the proximal force on IMD 110 when IMD 110 is within cup volume 112 (e.g., after cup volume 112 has received IMD 110) to, for example, cause delivery cup volume 132 of delivery catheter 128 to receive driver cup 106 and IMD 110. In examples, medical system 100 defines a loop (e.g., snare loop 176 (FIG. 2), tether loop 182 (FIGS. 10-13)) configured to engage IMD 110 (e.g., retrieval structure 140) to enable exertion of a proximal force (e.g., a proximal force exerted by a clinician).

In some examples, medical system 100 includes a snare 172 (FIG. 2) including an body 174 (“snare body 174”) supporting a loop 176 (“snare loop 176”). Snare body 174 may be an elongate body defining a proximal portion 178 (“snare proximal portion 178”) and a distal portion 180 (“snare distal portion 180”). Snare 172 may support snare loop 176 with snare distal portion 180. In examples, snare proximal portion 178 is configured to be extracorporeal to a patient when at least some portion of snare distal portion 180 and/or snare loop 176 is intracorporeal to the patient.

Snare loop 176 is configured to engage IMD 110 (e.g., retrieval structure 140). Snare 172 is configured such that a proximal force (e.g., in the proximal direction P) exerted on snare body 174 (e.g., by a clinician) causes snare loop 176 to exert the proximal force on IMD 110 when snare loop 176 is engaged with IMD 110. In examples, at least snare body 174 is configured to slidably translate with a lumen of medical system 100 (e.g., member lumen 159, driver lumen 124, and/or delivery catheter lumen 141) to cause snare loop 176 to exert the proximal force on IMD 110. Some portion of or substantially all of snare loop 176 may be configured to slidably translate with the lumen of medical system 100 when snare body 174 translates in the lumen. For example, snare loop 176 is configured to at least partially collapse to slidably translate with the lumen of medical system 100. In examples, snare body 174 is sufficiently flexible to define a curved and/or curvilinear shape within the lumen of medical system 100. In some examples, snare body 174 may be sufficiently rigid to cause snare loop 176 to extend distally beyond driver cup opening 131 to engage with (e.g., capture) IMD 110. Snare loop 176 and/or snare body 174 may be resiliently biased such that snare loop 176 substantially establishes a particular orientation relative to snare body 174 when snare loop 176 is unconstrained by the lumen of the medical system. For example, snare loop 176 and/or snare body 174 may be resiliently biased such that snare loop 176 and snare body 174 define an angle (e.g., an angle of about 90 degrees, about 45 degrees, or some other angle) when snare loop 176 is unconstrained by the lumen of the medical system.

In some examples, intermediate member 122 includes the loop configured to engage IMD 110. For example, FIG. 10 illustrates intermediate member 122 (e.g., member body 119) supporting a tether loop 182. FIG. 11 illustrates medical system 100 with tether loop 182 engaging IMD 110 (e.g., retrieval structure 140).

Intermediate member 122 may support tether loop 182 using intermediate member distal portion 121 and/or intermediate member distal end 157. Tether loop 182 is configured such that a proximal force (e.g., in the proximal direction P) exerted on member body 119 (e.g., by a clinician) causes tether loop 182 to exert the proximal force on IMD 110 when tether loop 182 is engaged with IMD 110 (e.g., as depicted in FIG. 11). Tether loop 182 may be configured to engage IMD 110 in the same or a similar manner to that described for snare loop 176. Tether loop 182 may be configured with respect to member body 119 in the same or a similar manner as the configuration of snare loop 176 with respect to snare body 174. For example, member body 119 may be configured to cause tether loop 182 to exert the proximal force on IMD 110 when member body slidably translates in the proximal direction P within driver lumen 124. Some portion of or substantially all of tether loop 182 may be configured to slidably translate within driver lumen 124 when member body 119 translates within driver lumen 124. In some examples, member body 119 may be sufficiently rigid to cause tether loop 182 to extend distally beyond driver cup opening 131 to engage with (e.g., capture) IMD 110. In examples, tether loop 182 and/or member body 119 may be resiliently biased such that tether loop 182 substantially establishes a particular orientation (e.g., an angle of about 90 degrees, about 45 degrees, or some other angle) relative to member body 119 when tether loop 182 is unconstrained by driver lumen 124.

As discussed, driver body 104 is configured to transmit a torque to driver cup 106 (e.g., a torque around longitudinal axis L) to cause a rotation of driver cup 106 (e.g., about longitudinal axis L). Driver body 104 may be mechanically connected to driver cup 106 in any manner which establishes a rotational coupling between driver body 104 and driver cup 106, such as by welding, soldering, adhesives, pins, or some other suitable fastening method. In some examples, driver body 104 includes a torque coil 188. Torque coil 188 may have the form of a helix substantially surrounding a helix interior. Torque coil 188 may be configured within medical system 100 such that longitudinal axis L passes through at least some portion of the helix interior. In some examples, the helix interior defines at least part of driver lumen 124.

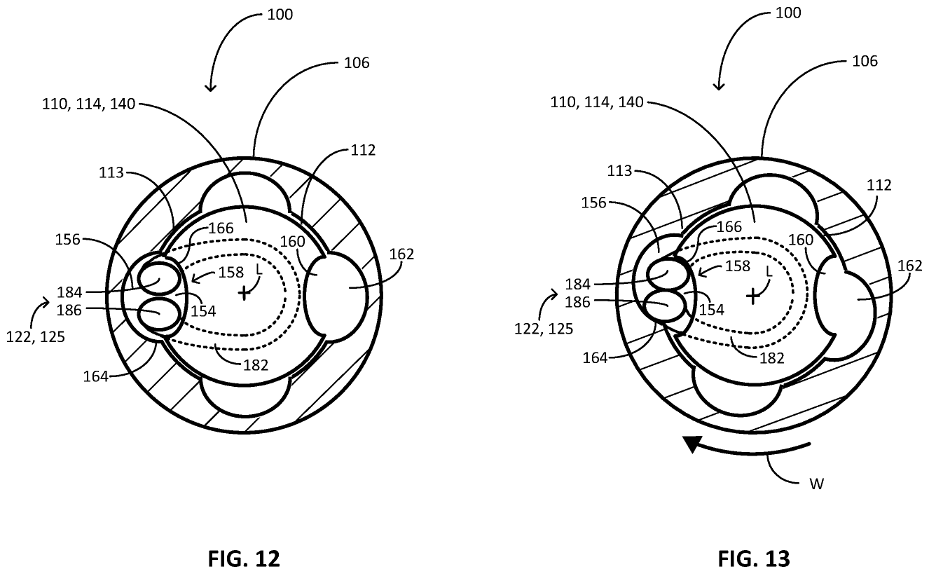

In examples, some portion of tether loop 182 defines transfer portion 125 of intermediate member 122. For example, FIG. 12 illustrates a cross-sectional view of medical system 100 taken along a cutting plane similar to cutting plane A of FIG. 5. FIG. 12 illustrates tether loop 182 engaged with IMD 110 (e.g., retrieval structure 140). In FIG. 12, tether loop 182 is illustrated engaging IMD 110 in dashed lines for clarity. Tether loop 182 is configured such that a first portion 184 of tether loop 182 (“first loop portion 184”) and/or a second portion 186 of tether loop 182 (“second loop portion 186”) may position within cup recess 156 and/or device recess 154 when tether loop 182 engages IMD 110. Tether loop 182 is configured such that, when first loop portion 184 and/or second loop portion 186 are positioned within cup recess 156 and device recess 154 and driver cup 106 imparts a torque on first loop portion 184 and/or second loop portion 186, first loop portion 184 and/or second loop portion 186 impart the torque to IMD 110. First loop portion 184 and/or second loop portion 186 may be configured individually or collectively relative to driver cup 106 in the same or a similar manner as the configuration of intermediate member 122 and/or transfer portion 125 is configured relative to driver cup 106. For example, cup recess surface 164 may be configured to impart a force on first loop portion 184 and/or second loop portion 186 when driver cup 106 rotates around longitudinal axis L. First loop portion 184 and/or second loop portion 186 may be configured to exert the force on device recess surface 166 to transfer the torque from driver cup 106 to IMD 110. First loop portion 184 and/or second loop portion 186 may define protruding portion 158.

For example, FIG. 13 illustrates driver cup 106 experiencing a torque (e.g., imparted from driver body 104 (FIG. 5) causing a rotation of driver cup 106 in the rotational direction W. The rotation of driver cup 106 causes cup recess surface 164 to contact and impart a force on first loop portion 184 and/or second loop portion 186. In examples, the force imparted from cup recess surface 164 to first loop portion 184 and/or second loop portion 186 comprises at least a force component substantially perpendicular to longitudinal axis L. In response, first loop portion 184 and/or second loop portion 186 contacts and transmits at least some portion of the force (e.g., a torquing force) to device recess surface 166. The portion of the force transmitted to device recess surface 166 causes a resultant torque on IMD 110 around longitudinal axis L. Hence, the action of first loop portion 184 and/or second loop portion 186 transmitting a force from cup recess surface 164 to device recess surface 166 acts to transfer a torque from driver cup 106 to IMD 110.

Operating circuitry 150 may include fixed function circuitry and/or programmable operating circuitry. In examples, operating circuitry 150 may include circuitry configured to perform one or more functions of operating circuitry 150, such as therapy delivery circuitry, sensing circuitry, processing circuitry, switching circuitry, communication circuitry, and/or other circuitries. Operating circuitry 150, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, operating circuitry 150 includes multiple components, such as any combination of one or more microprocessors, one or more DSPs, one or more ASICs, or one or more FPGAs, as well as other discrete or integrated logic circuitry, and/or analog circuitry.

Functions attributed to operating circuitry 150 may be embodied as software, firmware, hardware or any combination thereof. Operating circuitry 150 may include, for instance, a variety of capacitors, transformers, switches, and the like configured to perform the functions of operating circuitry 150. In examples, operating circuitry 150 may be configured to communicate with another device, such as a patient input/output device, a clinician input/output device, and/or others. Operating circuitry 150 may include any suitable hardware, firmware, software or any combination thereof for communicating with another device. In addition, operating circuitry 150 may communicate with a networked computing device and a computer network. In examples, operating circuitry 150 and/or other circuitry of medical system 100 is configured to deliver stimulation signals to and/or receive sensing signals from electrodes 142, 144, 148 and/or other electrodes and/or sensors within medical system 100 or external to medical system 100. Operating circuitry 150 may be configured to provide electrical signals, e.g., pacing therapy, to electrodes 142, 144, 148. Operating circuitry 150 may be configured to receive electrical signals, e.g., sensed cardiac electrical signals, from electrodes 142, 144, 148.

Medical system 100 (e.g., operating circuitry 150) can also include memory configured to store program instructions, such as software, which may include one or more program modules, which are executable by operating circuitry 150. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media. In some examples, the memory includes computer-readable instructions that, when executed by operating circuitry 150 cause operating circuitry 150 to perform various functions described herein and/or other functions of operating circuitry 150.

IMD housing 146 may enclose operating circuitry 150 and/or other circuitry within medical system 10. IMD housing 146 may be configured to fluidly isolate operating circuitry 150 and/or other circuitry from an environment in contact with an exterior surface of IMD housing 146. In examples, IMD housing 146 is configured to hermetically seal an enclosure defined by IMD 110 and holding operating circuitry 150 and/or other circuitry. IMD housing 146 may be configured to define shapes that are easily accepted by the patient's body while minimizing patient discomfort. For example, IMD housing 146 may define a substantially cylindrical shape with cylindrical sidewalls. In other examples, IMD housing 146 may define substantially rectangular or other non-cylindrical shapes. IMD housing 146 may define shapes in which corners and edges are designed with relatively large radii, in order to present a housing having smoothly contoured exterior surfaces. In examples, attachment member 118 is coupled to IMD housing 146.

As used here, when a first portion of a system (e.g., medical system 100) supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

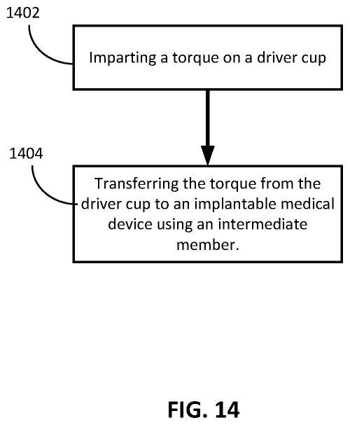

A technique for imparting a torque using a medical system 100 is illustrated in FIG. 14. Although the technique is described mainly with reference to medical system 100 of FIGS. 1-13, the technique may be applied to other medical systems in other examples.

The technique includes imparting a torque, using a driver body 104 of a driver 102, on a driver cup 106 supported at a driver body distal end 108 (1402). Driver body 104 may impart the torque on driver cup 106 to cause driver cup to rotate within an anatomical volume (e.g., a heart chamber) defined by a patient. In examples, the technique includes, using driver body 104, positioning a transfer portion 125 of an intermediate member 122 extending through a driver lumen 124 and a driver lumen opening 126 defined by driver 102. Driver body 104 may position transfer portion 125 within a cup recess 156 of driver cup 106. Driver body 104 may position transfer portion 125 within a device recess 154 of an IMD 110. IMD 110 may define device recess 154 with an IMD proximal portion 114 and/or a retrieval structure 140.

The technique may include engaging IMD 110 with a loop of medical system 100 configured to cause a proximal force on IMD 110. In some examples, a snare 172 including a snare loop 176 engages IMD 110 using snare loop 176. Snare loop 176 may exert the proximal force on IMD 110 when a proximal force is exerted (e.g., by a clinician) on a snare body 174 supporting snare loop 176. In examples, snare body 174 and/or snare loop 176 extend through a member lumen 159 and a member lumen opening 161 defined by intermediate member 122. Snare loop 176 may at least partially collapse (e.g., around IMD 110) when snare body 174 slidably translates within member lumen 159. In some examples, intermediate member 122 engages IMD 110 using a tether loop 182. Tether loop 182 may exert the proximal force on IMD 110 when a proximal force is exerted (e.g., by a clinician) on a member body 119 supporting tether loop 182. Tether loop 182 may at least partially collapse (e.g., around IMD 110) when member body 119 slidably translates within driver lumen 124.

Tether loop 182 or snare loop 176 may cause a cup volume 112 defined by driver cup 106 (e.g., defined by a cup surface 113) to receive IMD 110 (e.g., IMD proximal portion 114) when the proximal force is exerted. In examples, IMD 110 moves proximally (e.g., in a proximal direction P) relative to driver cup 106 to cause cup volume 112 to receive IMD 110. In examples, driver cup 106 moves distally (e.g., in a distal direction D) relative to IMD 110 to cause cup volume 112 to receive IMD 110.

In examples, driver cup 106 causes transfer portion 125 to position within cup recess 156 and/or device recess 154. Driver cup 106 may rotate relative to IMD 110 to position transfer portion 125 within cup recess 156 and/or device recess 154. In examples, rotation of driver cup 106 relative to IMD 110 and/or other portions of medical system 100 causes intermediate member 122 to insert (e.g., slot into) cup recess 156. In examples, rotation of driver cup 106 relative to IMD 110 and/or other portions of medical system 100 causes intermediate member 122 to insert (e.g., slot into) device recess 154. In some examples, rotation of driver cup 106 relative to IMD 110 and/or other portions of medical system 100 causes intermediate member 122 to slip between cup surface 113 and IMD 110 until intermediate member 122 slots into cup recess 156 and/or device recess 154. In some examples, tether loop 182 causes a first loop portion 184 and/or a second loop portion 186 to insert within device recess 154 when tether loop 182 engages IMD 110. First loop portion 184 and/or second loop portion 186 may define transfer portion 125.

The technique includes transferring the torque from driver cup 106 to IMD 110 using transfer portion 125 (1404). In examples, transfer portion 125 defines a protruding portion 168 protruding from cup recess 156 when transfer portion positions in cup recess 156. In examples, protruding portion 168 positions in device recess 154. Driver cup 106 may transfer torque to IMD 110 using protruding portion 168. In examples, transfer portion 125 revolves around a longitudinal axis L defined by driver body 104 when transfer portion 125 transfers the torque to IMD 110. In examples, cup recess surface 164 imparts a force from cup recess 156 to transfer portion 125 when driver body 104 imparts the torque on driver cup 106. Transfer portion 125 may transmit at least some portion of the force from transfer portion 125 to device recess surface 166 to transfer the torque from driver cup 106 to IMD 110.

Driver cup 106 may cause IMD 110 to rotate about a device axis LD defined by IMD 110 when transfer portion 125 transfers the torque from driver cup 106 to IMD 110. In examples, driver cup 106 causes an attachment member 118 of IMD 110 to disengage from tissue within or in proximity to a target site 120 within the patient when driver cup 106 causes IMD 110 to rotate about a device axis LD. In examples, driver cup 106 causes attachment member 118 of IMD 110 to engage tissue within or in proximity to target site 120 when driver cup 106 causes IMD 110 to rotate about device axis LD. In examples, driver cup 106 causes one or more device components of IMD 110 to rotate about device axis LD when driver cup 106 causes IMD 110 to rotate about device axis LD.

The technique may include positioning, using a delivery catheter 128, at least a portion of driver body 104, driver cup 106, and/or intermediate member 122 within the anatomical volume of the patient. In examples, delivery catheter 128 positions at least a portion of driver body 104, driver cup 106, and/or intermediate member 122 within a chamber of a heart of the patient. Delivery catheter 128 may transport at least a portion of driver body 104, driver cup 106, and/or intermediate member 122 through vasculature of the patient. In examples, at least driver cup 106 positions within a delivery cup volume 132 defined by delivery catheter 128. Driver body 104 may cause driver cup 106 to position within delivery cup volume 132 by slidably translating through a delivery catheter lumen 141 defined by delivery catheter 128.

The present disclosure includes (without limitation) the following examples.

Example 1. A medical system, comprising: a driver including a driver body configured to receive a torque, wherein the driver body defines a lumen and a lumen opening; a driver cup supported at a distal end of the driver body, wherein the driver body is configured to impart the torque on the driver cup, and wherein the driver cup includes a cup surface defining a cup volume configured to receive an implantable medical device, wherein the lumen opening opens into the cup volume; and an intermediate member including a member body and a transfer portion of the member body, wherein the member body is configured to slidably translate within the lumen to position the transfer portion within the cup volume, wherein the transfer portion is configured to position within a cup recess of the cup surface and a device recess of the implantable medical device when the cup volume receives the implantable medical device and the transfer portion is positioned within the cup volume, wherein the transfer portion is configured to transfer the torque from the driver cup to the implantable medical device when the transfer portion positions within the cup recess and the device recess, and wherein the driver body, the driver cup, and the intermediate member are configured to be positioned within an anatomical volume defined by a body of a patient.

Example 2. The medical system of Example 1, wherein the driver body, the driver cup, and the intermediate member are configured to be positioned within a chamber of a heart of the patient.

Example 3. The medical system of Example 1 or Example 2, wherein the driver body, the driver cup, and the intermediate member are configured to travel through vasculature of the patient.

Example 4. The medical system of any of Examples 1-3, wherein the transfer portion defines a cross-sectional dimension, and where the cross-sectional dimension causes a portion of the transfer portion to protrude from the cup recess when the transfer portion positions within the cup recess.

Example 5. The medical system of Example 4, wherein the protruding portion of the transfer portion is configured to transfer the torque from the driver cup to the implantable medical device.

Example 6. The medical system of any of Examples 1-5, wherein the intermediate member defines a first cross-sectional dimension, and wherein the cup surface includes a cup recess surface defining a second cross-sectional dimension, wherein the second cross-sectional dimension is greater than the first cross-sectional dimension.

Example 7. The medical system of any of Examples 1-6, wherein the driver body defines a longitudinal axis extending through the cup volume, and wherein the cup recess is configured to cause the transfer portion to revolve around the longitudinal axis when the transfer portion transfers the torque.

Example 8. The medical system of any of Examples 1-7, wherein the cup recess is configured to impart a force substantially perpendicular to the longitudinal axis on the transfer portion when the transfer portion transfers the torque.

Example 9. The medical system of Example 8, wherein the protruding portion of Example 5 is configured to impart a torquing force on the device recess when the cup recess imparts the force on the transfer portion and the transfer portion positions within the cup recess and the device recess.

Example 10. The medical system of Example 9, wherein the torquing force defines a force component parallel to the force imparted by the cup recess on the transfer portion.

Example 11. The medical system of any of Examples 1-10, wherein the member body defines a flexible portion contiguous with and proximal to the transfer portion, wherein the transfer portion defines a first stiffness and the flexible portion defines a second stiffness, wherein the first stiffness is greater than the second stiffness.

Example 12. The medical system of any of Examples 1-11, further comprising a snare defining a snare loop configured to engage the implantable medical device to exert at least a proximal force on the implantable medical device, wherein the snare is configured to slidably translate within the lumen.

Example 13. The medical system of any of Examples 1-12, wherein the intermediate member defines a member lumen extending through the transfer portion and defines a member lumen opening in the transfer potion, wherein the member lumen opening is configured to open to the cup volume when the when the transfer portion positions within the cup recess.

Example 14. The medical system of Example 13, wherein the snare of Example 12 is configured to slidably translate within the member lumen.

Example 15. The medical system of any of Examples 1-14, wherein the member body defines a distal portion and a tether loop at a distal end of the distal portion, wherein the tether loop is configured to engage the implantable medical device to exert at least a proximal force on the implantable medical device, and wherein at least one of the distal portion or the tether loop defines the transfer portion.

Example 16. The medical system of Example 15, wherein the distal portion is configured to impart the proximal force to the tether loop.

Example 17. The medical system of any of Examples 1-16, further comprising a delivery catheter having a delivery cup, wherein the delivery cup defines a delivery cup volume configured to receive at least a portion of the implantable medical device, wherein the delivery catheter defines a delivery lumen and a delivery lumen opening, wherein the delivery lumen opening opens into the delivery cup volume, and wherein the driver body is configured to slidably translate within the delivery lumen and pass through the delivery lumen opening.

Example 18. The medical system of Example 17, wherein the delivery cup volume is configured to receive the driver cup.

Example 19. The medical system of Example 17 or Example 18, wherein the delivery cup volume is configured to receive the driver cup when the transfer portion positions within the cup recess.

Example 20. The medical system of any of Examples 17-19, wherein the driver cup is configured to rotate relative to the delivery cup when the delivery cup volume receives the driver cup.

Example 21. The medical system of any of Examples 17-20, wherein the driver body is configured to rotate relative to the delivery cup when the driver body is positioned within the delivery lumen.

Example 22. The medical system of any of Examples 1-21, further comprising the implantable medical device.

Example 23. The medical system of Example 22, wherein the driver cup is configured to rotate relative to the implantable medical device to cause the transfer portion to position within the device recess when the cup volume receives the implantable medical device.

Example 24. The medical system of Example 21 or Example 22, wherein the implantable medical device includes a retrieval structure in a proximal portion of the implantable medical device, wherein the driver cup is configured to receive at least some portion of the retrieval structure.

Example 25. The medical system of Example 24, wherein the retrieval structure defines the device recess.

Example 26. The medical system of any of Examples 21-25, wherein the implantable medical device is configured to rotate about a device axis defined by the implantable medical device when the transfer portion transfers the torque from the driver cup to the implantable medical device.

Example 27. The medical system of Example 26, wherein the implantable medical device includes an attachment member configured to at least one of engage tissue or disengage from tissue when the implantable medical device rotates about the device axis.

Example 28. The medical system of Example 26 or Example 27, wherein the implantable medical device supports one or more device components configured to rotate about the device axis when the implantable medical device rotates about the device axis.

Example 29. A method, comprising: imparting a torque, using a driver body of a driver, on a driver cup supported at a distal end of the driver body, wherein the driver body defines a lumen and a lumen opening; and transferring the torque from the driver cup defining a cup volume to an implantable medical device within the cup volume using a transfer portion of an intermediate member positioned within a cup recess and a device recess, wherein the lumen opening opens into the cup volume, wherein the cup recess is defined by the driver cup and the device recess is defined by the implantable medical device, wherein the intermediate member includes a member body including the transfer portion, wherein the member body is slidably translatable within the lumen, and wherein the driver body, the driver cup, and the intermediate member are positioned within an anatomical volume defined by a body of a patient.

Example 30. The method of Example 29, further comprising transferring the torque to the implantable medical device using a portion of the transfer portion protruding from the cup recess when the transfer portion positions within the cup recess.

Example 31. The method of Examples 29 or Example 30, further comprising causing the transfer portion to revolve around a longitudinal axis defined by the driver body when the transfer portion transfers the torque.

Example 32. The method of any of Examples 29-31, further comprising: imparting a force from the cup recess to the transfer portion when the driver body imparts the torque on the driver cup; and transmitting at least some portion of the force from the transfer portion to the device recess to transfer the torque to the implantable medical device.

Example 33. The method of any of Examples 29-32, further comprising engaging the implantable medical device to exert at least a proximal force on the implantable medical device using a snare loop defined by a snare, wherein the snare is configured to slidably translate within the lumen.

Example 34. The method of any of Examples 29-33, further comprising slidably translating the snare of Example 33 through a member lumen defined by the intermediate member, wherein the member lumen extends through the transfer portion and open to the cup volume.

Example 35. The medical system of any of Examples 29-34, wherein the member body defines a defines a distal portion and a tether loop at a distal end of the distal portion, and further comprising positioning at least one of the distal portion or the tether loop within the cup recess and the device recess, such that the at least one of the distal portion of the tether loop defines the transfer portion.

Example 36. The method of any of Examples 29-35, further comprising engaging the implantable medical device to exert at least a proximal force on the implantable medical device using the tether loop of Example 35.

Example 37. The method of any of Examples 29-36, further comprising positioning the driver cup within a delivery cup volume defined by a delivery cup of a delivery catheter by slidably translating the driver body through a delivery lumen defined by the delivery catheter.

Example 38. The method of any of Examples 29-37, further comprising positioning, using the delivery catheter of Example 37, the driver body, the driver cup, and the intermediate member within a chamber of a heart of the patient.

Example 39. The method of any of Examples 29-38, further comprising transporting, using the delivery catheter of Example 37, the driver body, the driver cup, and the intermediate member through vasculature of the patient.

Example 40. The method of any of Examples 29-39, further comprising rotating the driver cup relative to the delivery cup of Example 37 when the driver cup is positioned within the delivery cup volume of Example 37.

Example 41. The method of any of Examples 29-40, further comprising rotating the driver body relative to the delivery cup of Example 37 to impart the torque on the driver cup.

Example 42. The method of Examples 29-41, further comprising rotating the driver cup relative to the implantable medical device to cause the transfer portion to position within the device recess.

Example 43. The method of Examples 29-42, further comprising receiving a retrieval structure of the implantable medical device within the cup volume.

Example 44. The method of Examples 29-43, further comprising causing, using the torque imparted by the driver body, the implantable medical device to rotate about a device axis defined by the implantable medical device.

Example 45. The method of Example 44, further comprising causing an attachment member of the implantable medical device to at least one of engage tissue or disengage from tissue when the implantable medical device rotates about the device axis.

Example 46. The method of Example 44 or Example 45, further comprising causing one or more device components to rotate about the device axis when the implantable medical device rotates about the device axis to cause the one or more device components to achieve a particular orientation relative to the body of the patient.

Example 47. A medical system, comprising: a driver receptacle which is rotatable with respect to a longitudinal axis of the medical system, the driver receptacle including a receptacle surface defining a receptacle volume configured to receive at least a proximal portion of an implantable medical device; and an intermediate member including a member body and a transfer portion of the member body, wherein the member body is configured to longitudinally translate relative to the receptacle to position the transfer portion along the receptacle volume, wherein the transfer portion is configured to position within a receptacle recess of the receptacle surface when the receptacle volume receives the implantable medical device and the transfer portion is positioned within the receptacle volume, wherein the transfer portion is configured to transfer the torque from the driver receptacle to the implantable medical device when the transfer portion positions within the receptacle recess, and wherein the driver body, the driver receptacle, and the intermediate member are configured to be positioned within an anatomical volume defined by a body of a patient.

Example 48. The medical system of Example 47, wherein the driver receptacle and the intermediate member are configured to be positioned within a chamber of a heart of the patient.

Example 49. The medical system of Example 47 or Example 48, wherein the driver receptacle and the intermediate member are configured to travel through vasculature of the patient.

Example 50. The medical system of any of Examples 47-49, wherein the transfer portion defines a cross-sectional dimension, and where the cross-sectional dimension causes a portion of the transfer portion to protrude from the receptacle recess when the transfer portion positions within the receptacle recess.

Example 51. The medical system of Example 50, wherein the protruding portion of the transfer portion is configured to transfer the torque from the driver receptacle to the implantable medical device.

Example 52. The medical system of any of Examples 47-51, wherein the intermediate member defines a first cross-sectional dimension, and wherein the receptacle surface includes a receptacle recess surface defining a second cross-sectional dimension, wherein the second cross-sectional dimension is greater than the first cross-sectional dimension.

Example 53. The medical system of any of Examples 47-52, wherein the receptacle recess is configured to cause the transfer portion to revolve around the longitudinal axis when the transfer portion transfers the torque.

Example 54. The medical system of any of Examples 47-53, wherein the receptacle recess is configured to impart a force substantially perpendicular to the longitudinal axis on the transfer portion when the transfer portion transfers the torque.

Example 55. The medical system of Example 54, wherein: the implantable medical device further comprises a device recess; the transfer portion is configured to position within the receptacle recess and the device recess when the receptacle volume receives the implantable medical device and the transfer portion is positioned within the receptacle volume; and the protruding portion of Example 5 is configured to impart a torquing force on the device recess when the receptacle recess imparts the force on the transfer portion and the transfer portion positions within the receptacle recess and the device recess.

Example 56. The medical system of Example 55, wherein the torquing force defines a force component parallel to the force imparted by the receptacle recess on the transfer portion.

Example 57. The medical system of any of Examples 47-56, wherein the member body defines a flexible portion contiguous with and proximal to the transfer portion, wherein the transfer portion defines a first stiffness and the flexible portion defines a second stiffness, wherein the first stiffness is greater than the second stiffness.

Example 58. The medical system of any of Examples 47-57, further comprising a snare defining a snare loop configured to engage the implantable medical device to exert at least a proximal force on the implantable medical device, wherein the snare is configured to slidably translate within the lumen.

Example 59. The medical system of any of Examples 47-58, wherein the intermediate member defines a member lumen extending through the transfer portion and defines a member lumen opening in the transfer portion, wherein the member lumen opening is configured to open to the receptacle volume when the when the transfer portion positions within the receptacle recess.

Example 60. The medical system of Example 59, wherein the snare of Example 12 is configured to slidably translate within the member lumen.

Example 61. The medical system of any of Examples 47-60, wherein the member body defines a distal portion and a tether loop at a distal end of the distal portion, wherein the tether loop is configured to engage the implantable medical device to exert at least a proximal force on the implantable medical device, and wherein at least one of the distal portion or the tether loop defines the transfer portion.

Example 62. The medical system of Example 61, wherein the distal portion is configured to impart the proximal force to the tether loop.

Example 63. The medical system of any of Examples 47-62, further comprising a delivery catheter having a delivery receptacle, wherein the delivery receptacle defines a delivery receptacle volume configured to receive at least a portion of the implantable medical device, wherein the delivery catheter defines a delivery lumen and a delivery lumen opening, wherein the delivery lumen opening opens into the delivery receptacle volume, and wherein the driver receptacle is configured to slidably translate within the delivery lumen and pass through the delivery lumen opening.

Example 64. The medical system of Example 63, wherein the delivery receptacle volume is configured to receive the driver receptacle.

Example 65. The medical system of Example 63 or Example 64, wherein the delivery receptacle volume is configured to receive the driver receptacle when the transfer portion positions within the receptacle recess.

Example 66. The medical system of any of Examples 63-65, wherein the driver receptacle is configured to rotate relative to the delivery receptacle when the delivery receptacle volume receives the driver receptacle.

Example 67. The medical system of any of Examples 63-66, further comprising a driver body extending proximally from the driver receptacle, the driver body being configured to rotate relative to the delivery receptacle when the driver body is positioned within the delivery lumen.

Example 68. The medical system of any of Examples 47-67, further comprising the implantable medical device.

Example 69. The medical system of Example 68, wherein the driver receptacle is configured to rotate relative to the implantable medical device to cause the transfer portion to position within the device recess when the receptacle volume receives the implantable medical device.

Example 70. The medical system of Example 67 or Example 68, wherein the implantable medical device includes a retrieval structure in a proximal portion of the implantable medical device, wherein the driver receptacle is configured to receive at least some portion of the retrieval structure.

Example 71. The medical system of Example 70, wherein the retrieval structure defines the device recess.

Example 72. The medical system of any of Examples 47-71, wherein the implantable medical device is configured to rotate about a device axis defined by the implantable medical device when the transfer portion transfers the torque from the driver receptacle to the implantable medical device.

Example 73. The medical system of Example 72, wherein the implantable medical device includes an attachment member configured to at least one of engage tissue or disengage from tissue when the implantable medical device rotates about the device axis.

Example 74. The medical system of Example 72 or Example 73, wherein the implantable medical device supports one or more device components configured to rotate about the device axis when the implantable medical device rotates about the device axis.

Example 75. The medical system of Example 47, further comprising a driver body configured to impart torque to the driver receptacle, the driver body defining a lumen and a lumen opening which opens into the receptacle volume.

Example 76. A method, comprising imparting a torque to the implantable medical device using the medical system of any of Examples 47-75.

Example 77. The method of Example 76, wherein imparting torque comprises implanting the implantable medical device into cardiac tissue.

Example 78. The method of Example 76, wherein imparting torque comprises removing the implantable medical device from cardiac tissue.

Various examples of the disclosure have been described. Any combination of the described systems, operations, or functions is contemplated. These and other examples are within the scope of the following claims.

1. A medical system, comprising:
a driver receptacle which is rotatable with respect to a longitudinal axis of the medical system, the driver receptacle including a receptacle surface defining a receptacle volume configured to receive at least a proximal portion of an implantable medical device; and
an intermediate member including a member body and a transfer portion of the member body, wherein the member body is configured to longitudinally translate relative to the receptacle to position the transfer portion along the receptacle volume,
wherein the transfer portion is configured to position within a receptacle recess of the receptacle surface when the receptacle volume receives the implantable medical device and the transfer portion is positioned within the receptacle volume,
wherein the transfer portion is configured to transfer the torque from the driver receptacle to the implantable medical device when the transfer portion positions within the receptacle recess, and
wherein the driver body, the driver receptacle, and the intermediate member are configured to be positioned within an anatomical volume defined by a body of a patient.

2. The medical system of claim 1, wherein the driver receptacle and the intermediate member are configured to be positioned within a chamber of a heart of the patient.

3. The medical system of claim 1, wherein the driver receptacle and the intermediate member are configured to travel through vasculature of the patient.

4. The medical system of claim 1, wherein the transfer portion defines a cross-sectional dimension, wherein the cross-sectional dimension causes a portion of the transfer portion to protrude from the receptacle recess when the transfer portion positions within the receptacle recess.

5. The medical system of claim 4, wherein the protruding portion of the transfer portion is configured to transfer the torque from the driver receptacle to the implantable medical device.

6. The medical system of claim 1, wherein the intermediate member defines a first cross-sectional dimension, and wherein the receptacle surface includes a receptacle recess surface defining a second cross-sectional dimension, wherein the second cross-sectional dimension is greater than the first cross-sectional dimension.

7. The medical system of claim 1, wherein the receptacle recess is configured to cause the transfer portion to revolve around the longitudinal axis when the transfer portion transfers the torque.

8. The medical system of claim 1, wherein the receptacle recess is configured to impart a force substantially perpendicular to the longitudinal axis on the transfer portion when the transfer portion transfers the torque.

9. The medical system of claim 8, wherein:
the implantable medical device further comprises a device recess;
the transfer portion is configured to position within the receptacle recess and the device recess when the receptacle volume receives the implantable medical device and the transfer portion is positioned within the receptacle volume;
the transfer portion defines a cross-sectional dimension, wherein the cross-sectional dimension causes a portion of the transfer portion to protrude from the receptacle recess when the transfer portion positions within the receptacle recess; and
the protruding portion of the transfer portion is configured to impart a torquing force on the device recess when the receptacle recess imparts the force on the transfer portion and the transfer portion positions within the receptacle recess and the device recess.

10. The medical system of claim 9, wherein the torquing force defines a force component parallel to the force imparted by the receptacle recess on the transfer portion.

11. The medical system of claim 1, wherein the member body defines a flexible portion contiguous with and proximal to the transfer portion, wherein the transfer portion defines a first stiffness and the flexible portion defines a second stiffness, wherein the first stiffness is greater than the second stiffness.

12. The medical system of claim 1, further comprising a snare defining a snare loop configured to engage the implantable medical device to exert at least a proximal force on the implantable medical device, wherein the snare is configured to slidably translate within the lumen.

13. The medical system of claim 1, wherein the intermediate member defines a member lumen extending through the transfer portion and defines a member lumen opening in the transfer portion, wherein the member lumen opening is configured to open to the receptacle volume when the when the transfer portion positions within the receptacle recess.

14. The medical system of claim 13, wherein the snare of claim 12 is configured to slidably translate within the member lumen.

15. The medical system of claim 1, wherein the member body defines a distal portion and a tether loop at a distal end of the distal portion, wherein the tether loop is configured to engage the implantable medical device to exert at least a proximal force on the implantable medical device, and wherein at least one of the distal portion or the tether loop defines the transfer portion.

16. A medical system, comprising:
a driver including a driver body configured to receive a torque, wherein the driver body defines a lumen and a lumen opening;
a driver cup supported at a distal end of the driver body, wherein the driver body is configured to impart the torque on the driver cup, and wherein the driver cup includes a cup surface defining a cup volume configured to receive an implantable medical device, wherein the lumen opening opens into the cup volume; and
an intermediate member including a member body and a transfer portion of the member body, wherein the member body is configured to slidably translate within the lumen to position the transfer portion within the cup volume,
wherein the transfer portion is configured to position within a cup recess of the cup surface and a device recess of the implantable medical device when the cup volume receives the implantable medical device and the transfer portion is positioned within the cup volume,
wherein the transfer portion is configured to transfer the torque from the driver cup to the implantable medical device when the transfer portion positions within the cup recess and the device recess, and
wherein the driver body, the driver cup, and the intermediate member are configured to be positioned within an anatomical volume defined by a body of a patient.

17. The medical system of claim 16, wherein the transfer portion defines a cross-sectional dimension, and where the cross-sectional dimension causes a portion of the transfer portion to protrude from the cup recess when the transfer portion positions within the cup recess.

18. The medical system of claim 16, further comprising a snare defining a snare loop configured to engage the implantable medical device to exert at least a proximal force on the implantable medical device, wherein the snare is configured to slidably translate within the lumen.

19. A method, comprising:
imparting a torque, using a driver body of a driver, on a driver cup supported at a distal end of the driver body, wherein the driver body defines a lumen and a lumen opening; and
transferring the torque from the driver cup defining a cup volume to an implantable medical device within the cup volume using a transfer portion of an intermediate member positioned within a cup recess and a device recess,
wherein the lumen opening opens into the cup volume,
wherein the cup recess is defined by the driver cup and the device recess is defined by the implantable medical device,
wherein the intermediate member includes a member body including the transfer portion,
wherein the member body is slidably translatable within the lumen, and
wherein the driver body, the driver cup, and the intermediate member are positioned within an anatomical volume defined by a body of a patient.

20. The method of claim 19, further comprising transferring the torque to the implantable medical device using a portion of the transfer portion protruding from the cup recess when the transfer portion positions within the cup recess.
